(19) United States
(12) Patent Application Publication
(10) Pub. No.: US 2006/0129136 A1
(43) Pub. Date: Jun. 15, 2006

(54) Catheter

(76) Inventor: George Bevan Kirby Meacham, Cleveland, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 11/298,788

(22) Filed: Dec. 9, 2005

Related U.S. Application Data
(60) Provisional application No. 60/634,693, filed on Dec. 9, 2004.

(51) Int. Cl. A61M 1/00

(52) U.S. Cl. 604/540; 604/544

(57) ABSTRACT

An apparatus (50) is adapted for insertion in a body lumen (140) having a surgical connection (58). The apparatus (50) includes a first conduit (80) insertable in the lumen (140) and having a distal end portion spaced distally beyond the surgical connection (58). The first conduit (80) directs fluid through the lumen (140) and isolates the fluid from the surgical connection (58). The apparatus (50) also includes a second conduit (84) insertable in the lumen (140). The second conduit (84) has a distal end portion positioned adjacent the surgical connection (58). The second conduit (84) is adapted to control the pressure in a space (90) in which the surgical connection (58) is exposed.

### RELATED APPLICATIONS

This application claims the benefit of U.S. Provisional Application No. U.S. 60/634,693, filed Dec. 9, 2004.

### TECHNICAL FIELD

The present invention is related to systems, methods, and apparatus for reducing leakage through connections, such as sutured connections, in lumens in the body and drawing the connections together to promote quick and complete healing. More particularly, it is directed to catheters that facilitate healing of sutured connections such as those between the urethra and the bladder neck following radical prostatectomy.

### BACKGROUND

Many types of surgery require connections or repairs in body lumens that must carry fluid during the healing process. A catheter is often inserted in the lumen to reduce pressure on the connection and provide mechanical support during the healing process. When using a catheter in this manner, however, the fluid pressure balance is typically such that fluid may leak through the annular area between the catheter and the lumen. The fluid may then contact the connection, leak through the connection into the surrounding tissue, and retard healing of the connection. Radical prostatectomy is an example of such surgery. In this procedure, the prostate gland is removed together with the section of the urethra that passes through the gland. The remaining urethra is sutured to the bladder to provide a urine passage.

Typically, a catheter, such as one commonly referred to in the art as a “Foley” catheter, is inserted during surgery to provide urine drainage and promote healing. Also, one or more drains may be placed in the abdominal cavity to remove urine that may leak from the urethra through the sutured connection between the urethra and bladder. This method is shown in FIGS. 1-3. As shown in FIG. 1, a catheter 1 (e.g., a Foley catheter) is inserted into the body 2 through the penis and urethra 3, beyond the sutured connection 4, and into the bladder 5. Typically, the catheter 1 is constructed of flexible materials, such as latex or silicone rubber, and is supplied sterilized. The catheter 1 may be coated with hydrogel to reduce friction and improve comfort, and antibacterial agents may be added to reduce the risk of infection.

An elastic balloon or bulb 6 at the bladder or distal end of the catheter 1 is inflated or filled with saline solution to anchor it in the bladder 5. As best shown in FIGS. 2 and 3, the catheter 1 has a central urine conduit 7 that drains to an external collection bag (not shown) and a concentric saline tube or second conduit 8 that is used to inflate or fill the elastic bulb 6. Once the bulb 6 is filled, the second conduit 8 is capped off. An annular region 9 (FIG. 3) is defined between the catheter 1 and the sutured connection 4. Initially, the sutured connection 4 between the bladder 5 and the urethra 3 is not fluid-tight. As a result, urine may seep or leak between the bulb 6 and the bladder 5 into the annular region 9, through the sutured connection 4, and into the abdominal cavity 12 (FIG. 1) of the body 2. A drain tube 10, such as one commonly referred to in the art as a “Jackson-Pratt” drain, is passed through the abdominal wall 11. A compressed elastic squeeze bulb (not shown) is used as a suction pump to apply a slight vacuum P_{ab} to the abdominal cavity 12 and collect urine leakage through drain tube 10. Fluid in the squeeze bulb is periodically emptied and the bulb is re-compressed to maintain the vacuum. After healing of the sutured connection 4 has progressed to the point that the urine leakage flow stops, the drain tube 10 is removed.

The catheter 1 is left in place after surgery for a period that may, for example, last from 10 days to 4 weeks. This serves several purposes in the healing process. The catheter 1 continuously drains urine from the bladder 5 to assure complete voiding and reduce pressure stress and leakage at the sutured connection 4. Also, the catheter 1 provides a mechanical support to reduce shrinkage of the urine passage caused by scar tissue contraction as the sutured connection 4 between the bladder 5 and the urethra 3 heals. Further, the catheter 1 allows for the application of a traction force 14 that presses the bladder 5 against the urethra 3 to promote healing of the sutured connection 4.

In the catheterization shown in FIGS. 1-3, the bladder 5 is maintained at atmospheric pressure Pₐₜ by the open urine conduit 7 while the abdominal cavity 12 is maintained at a pressure P_{ab} below atmospheric pressure due to the vacuum. The pressure differential between Pₐₜ and P_{ab} can lead to the leakage described above, i.e., urine leakage or seepage around the elastic bulb 6, into the annular region 9, and through the sutured connection 4 into the abdominal cavity 12. Also, since fluid pressure forces 13 (FIG. 3) may tend to open gaps in the sutured connection 4, urine leakage through the sutured connection 4 may retard healing, thereby lengthening recovery. Mechanical traction forces 14 on the catheter 1 and elastic bulb 6 are helpful since they tend to close these gaps and at least partially counter the pressure forces 13. Traction is, however, difficult to maintain over extended periods, such as days to weeks, and can be uncomfortable.

### SUMMARY

The present invention is related to systems, methods, and apparatus for creating a favorable pressure balance across a sutured connection of a body lumen to promote healing. According to one embodiment, a concentric tube surrounds the catheter and provides an annular conduit extending from the vicinity of the sutured connection to a position outside of the body. A distal end of the concentric tube has an opening that provides fluid communication between the annular conduit and an annular region defined generally between the sutured connection and the catheter. A proximal end of the concentric tube communicates with a service connection. A partial vacuum can be applied via the service connection, creating a reduced pressure Pₐₙ in the annular region. Pₐₙ is set lower than atm ospheric pressure Pₐₜ and an abdominal cavity pressure P_{ab} to create a pressure differential across the sutured connection that creates pressure forces that tend to close the sutured connection to speed healing without mechanical traction and to intercept and remove fluid seepage or leakage into the annular region, thus preventing seepage or leakage through the healing sutured connection.

According to one aspect, the present invention relates to an apparatus adapted for insertion in a body lumen having a surgical connection. The apparatus includes a first conduit insertable in the lumen. The first conduit has a distal end portion for being positioned spaced distally beyond the surgical connection. The first conduit directs fluid through the lumen and isolates the fluid from the surgical connection. The apparatus also includes a second conduit insertable in the lumen. The second conduit has a distal end portion positioned adjacent the surgical connection. The second conduit is adapted to control the pressure in a space in which the surgical connection is exposed.

According to another aspect, the present invention relates to a catheter system that includes a Foley catheter insertable through a urethra into a bladder. The Foley catheter has a proximal end portion for being positioned outside the urethra and an opposite distal end portion for being disposed in the bladder. The Foley catheter includes a first conduit for emptying the bladder, an elastic bulb disposed on the distal end portion in the bladder, and a second conduit for delivering inflation fluid to the elastic bulb. The elastic bulb is inflatable to help retain the Foley catheter inserted in the bladder. The system also includes a concentric tubular member that encircles the Foley catheter. The tubular member has a proximal end portion positioned outside urethra and an opposite distal end portion that terminates before a proximal end of the elastic bulb. The tubular member defines a third conduit for controlling the pressure in a space defined between the tubular member and the bladder and urethra. A surgical connection between the bladder and urethra are exposed in the space.

According to a further aspect, the present invention relates to a method for facilitating the healing of a surgical connection in a body lumen. The method includes the step of providing a first conduit in the lumen. The method also includes the step of positioning a distal end portion of the first conduit distally beyond the surgical connection. The first conduit directs fluid through the lumen and isolates the fluid from the surgical connection. The method also includes the step of providing a second conduit in the lumen and positioning a distal end portion of the second conduit adjacent the surgical connection. The method further includes the step of regulating the pressure in a space in which the surgical connection is positioned via the second conduit.

### DRAWINGS

The foregoing and other features of the present invention will become apparent to those skilled in the art to which the present invention relates upon reading the following description with reference to the accompanying drawings, in which:

FIG. 1 illustrates a prior art urethra catheterization;

FIG. 2 is a sectional view taken generally along line 2-2 in FIG. 1;

FIG. 3 is a magnified view of a portion of FIG. 1;

FIG. 4 illustrates a catheterization system, method, and apparatus according to the present invention;

FIG. 5 is a sectional view taken generally along line 5-5 in FIG. 4; and

FIG. 6 is a magnified view of a portion of FIG. 4.

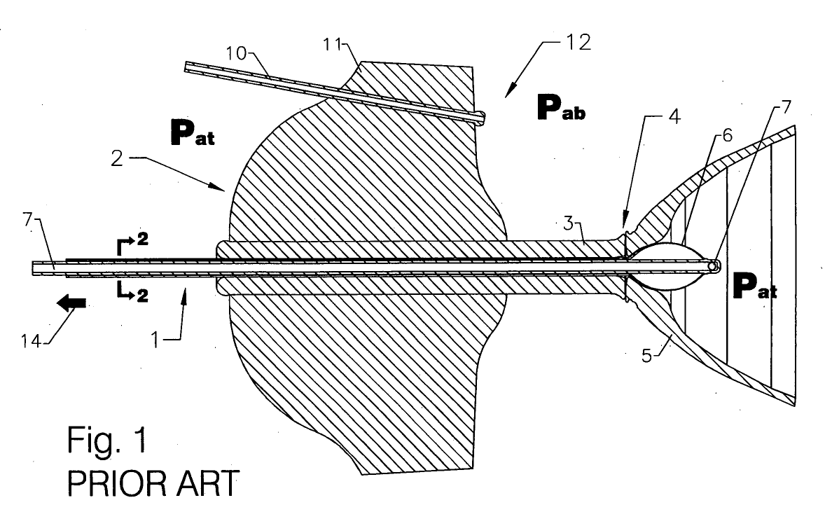

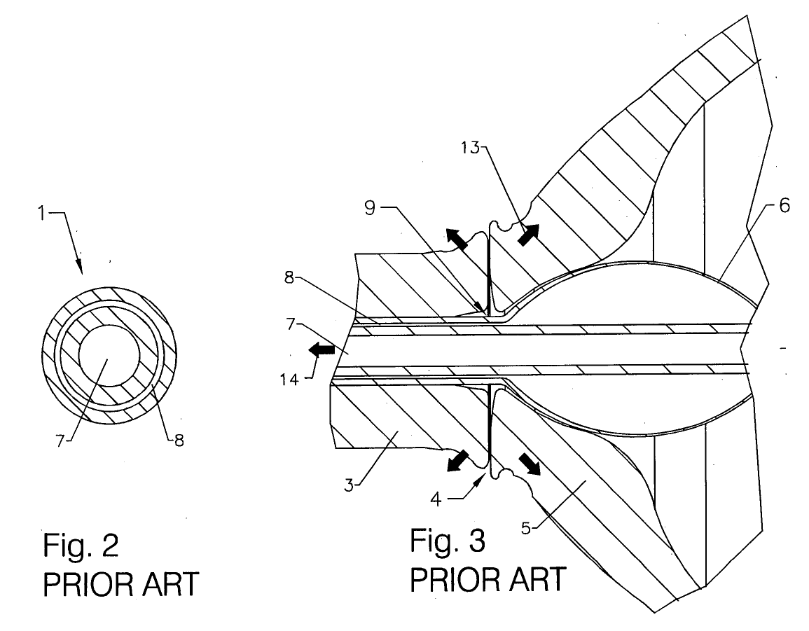

### DESCRIPTION

The present invention is related to systems, methods, and apparatus for helping to reduce leakage through sutured connections in lumens in the body and drawing the connections together to promote quick and complete healing. The invention is described herein with respect to urinary catheters to facilitate healing of a sutured connection between the urethra and the bladder following radical prostate prostatectomy surgery. It will, however be apparent to those skilled in the art that the following detailed description is similarly applicable to sutured connections of other body lumens and other types of surgery.

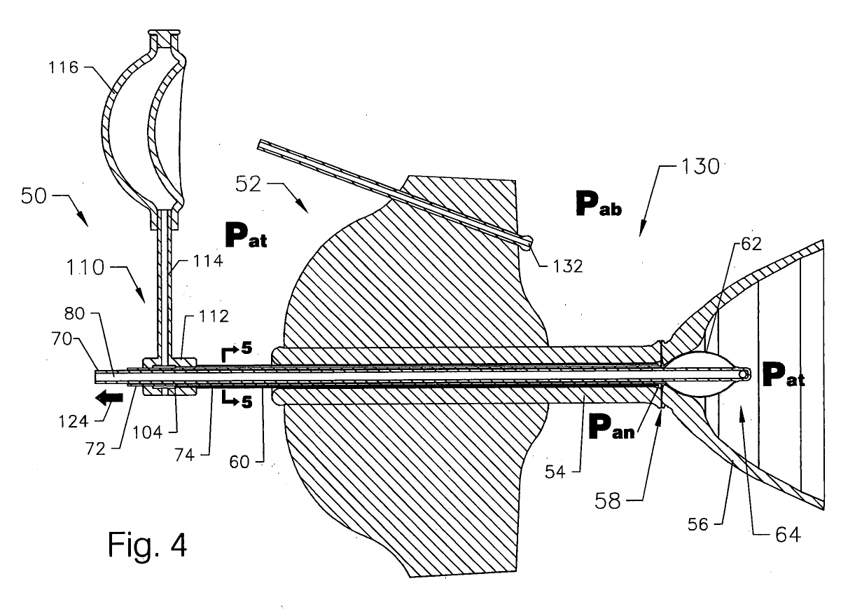

FIG. 4 is illustrative of a system or apparatus 50 for helping to reduce leakage through connections, such as sutured connections, in lumens in the body and drawing the connections together to facilitate and promote quick and complete healing. FIG. 4 illustrates a body 52 in which the prostate gland (not shown) together with the section of the urethra that passes through the gland is removed surgically. The remaining urethra 54 is attached to the bladder 56 via a surgical, e.g., sutured, connection 58 to provide a urine passage. The catheter 60 is inserted into the body 52 through the penis and urethra 54, beyond the sutured connection 58 and into the bladder 56.

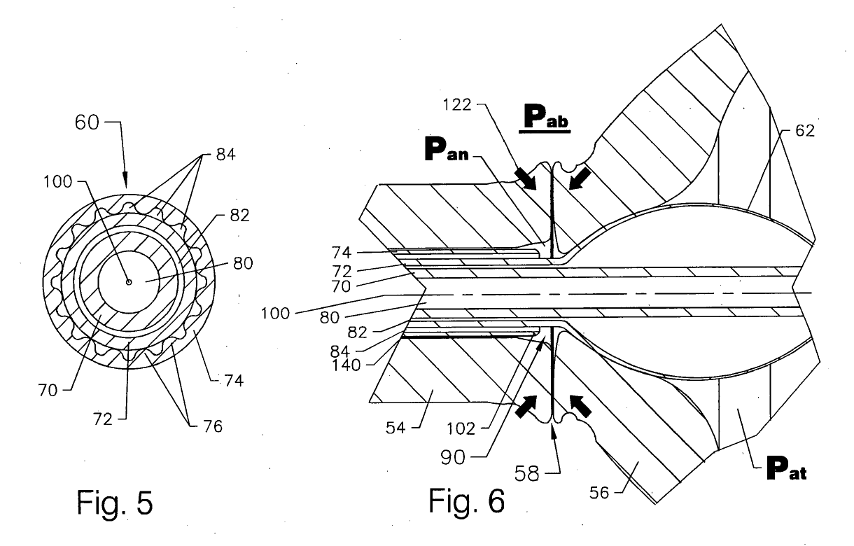

Referring to FIGS. 5 and 6, the catheter 60 includes an inner, first tube or tubular member 70, a middle, second tube or tubular member 72, and an outer, third tube or tubular member 74. The tubes 70, 72, and 74 are generally elongate and flexible and are constructed of materials, such as latex or silicone rubber, that are suitable for the medical uses described herein. The tubes 70, 72, and 74 are arranged generally concentric with each other and extend along a longitudinal axis 100.

The first tube 70 defines a first passage or conduit 80 that, in the embodiment illustrated and described herein, serves as a urine passage, which is described in further detail below. An annular second passage or conduit 82 is defined between an outer surface of the first tube 70 and an inner surface of the second tube 72. In the embodiment illustrated and described herein, the second conduit 82 serves as an inflation passage for inflating or filling an elastic bulb 62, which is described in further detail below. A third passage or conduit 84 is defined between an outer surface of the second tube 72 and an inner surface of the third tube 74. In the embodiment illustrated and described herein, the third tube 74 may include ribs 76 on its inner surface that help support and maintain the position of the third tube on the second tube 72. The third conduit 84 thus may comprise a series of axial grooves defined by the ribs 76 and spaced about the circumference of the second tube 72. Alternatively, the third conduit 84 could have an annular configuration similar to that of the second conduit 82. In the embodiment illustrated and described herein, the third conduit 84 serves as a vacuum passage for helping to control pressure differentials between an annular pressure Pₐₙ, an abdominal pressure P_{ab}, and atmospheric pressure Pₐₜ, which is described in further detail below.

When the catheter 60 is inserted as shown in FIGS. 4 and 6, the urine conduit 80 drains urine from the bladder to an external collection bag (not shown). The elastic bulb 62 at the bladder or distal end 64 of the catheter 60 is inflated or filled with saline solution delivered via the second conduit 82 to anchor it in the bladder 56. Once the bulb 62 is filled, the second conduit 72 is capped off.

An annular region 90 is defined between the catheter 60 and the region of the urethra 54 and bladder 56 at or adjacent the sutured connection 58. The third tube 74 has a distal end portion 102 that terminates adjacent or just behind the elastic bulb 62 (i.e., before a proximal end of the bulb) at or in the vicinity of the annular region 90. This places the third conduit 84 in fluid communication with the annular region 90.

Referring to FIG. 4, an outside or proximal end portion 104 of the third tube 74 is fitted with a service connection 110 that communicates with the third conduit 84. The service connection 110 includes an annular collar 112 that encircles the first and second conduits 70 and 72 and engages the third conduit 74 to establish fluid communication with the third conduit 84. The service connection 110 also includes a service tube or tap 114 that is connected to the collar 112 and is in fluid communication with the third conduit 84 via the collar 112.

In use, a partial vacuum is applied to the service connection 110. Any suitable vacuum source may be used, such as an elastic squeeze bulb 116 secured to the service tube 114. This reduces the pressure Pₐₙ in the annular region 90 (see FIG. 5). The vacuum is applied such that Pₐₙ is lower than atmospheric pressure Pₐₜ and the abdominal pressure P_{ab}. As a result, the pressure differential across the sutured connection 58 is reversed compared to that illustrated in FIG. 3. This is indicated generally in FIG. 6 by the pressure forces 122. As shown in FIG. 6, the pressure forces 122 would tend to close the sutured connection 58, which would help speed healing. This would also help reduce or eliminate the need for the application of mechanical traction forces (indicated generally at 124 in FIG. 4).

Also, the reversed pressure differential would allow urine seeping around the bulb 62 into the annular region 90 to be intercepted and removed via the third conduit 84, which helps prevent the seepage from flowing through the healing sutured connection 58 and into the abdominal cavity 130. This may help eliminate or reduce the need for Jackson-Pratt drains 132 for urine removal from the abdominal cavity 130, although drains may still be needed for removal of other fluids. If a Jackson-Pratt drain 132 is used, the vacuum is adjusted such that Pₐₙ is less than P_{ab}. The vacuum may thus be applied to the annular region 90 throughout substantial portion of the duration of use of the system 50. For example, the vacuum may be removed for periodic maintenance of the system 50.

In other respects, the catheter 60 of the present invention provides similar or identical functions as the Foley catheters described above. The catheter 60 It is left in place during the healing process to drain urine from the bladder 56 in order to assure complete voiding and reduce pressure stress at the sutured connection 58. The catheter 60 also maintains the bladder 56 at atmospheric pressure Pₐₜ by the open urine conduit 80. The catheter 60 also provides mechanical support to reduce shrinkage of the urine passage caused by scar tissue contraction as the sutured connection 58 heals. Optionally, traction forces 124 may be applied to press the bladder 56 more tightly against the urethra 54 to supplement the pressure forces 122. The catheter 60 may thus comprise a Foley catheter and a concentric sleeve in which the Foley catheter is disposed.

From the above description of the invention, those skilled in the art will perceive improvements, changes and modifications. Such improvements, changes and modifications within the skill of the art are intended to be covered by the appended claims.

What is claimed is:

1. An apparatus adapted for insertion in a body lumen having a surgical connection, the apparatus comprising:
a first conduit insertable in the lumen, the first conduit having a distal end portion for being positioned spaced distally beyond the surgical connection, the first conduit directing fluid through the lumen and isolating the fluid from the surgical connection;
a second conduit insertable in the lumen, the second conduit having a distal end portion positioned adjacent the surgical connection, the second conduit being adapted to control the pressure in a space in which the surgical connection is exposed.

2. The apparatus recited in claim 1, further comprising:
a first tubular member that defines the first conduit;
a second tubular member that defines the second conduit; and
a third tubular member that defines a third conduit, the first tubular member being disposed in the second tubular member and the third tubular member being disposed between the first and second tubular members.

3. The apparatus recited in claim 2, further comprising a retention device disposed at a distal end of the third tubular member.

4. The apparatus recited in claim 3, wherein the retention device comprises an inflatable elastic bulb, the third conduit being for delivering a fluid for inflating the elastic bulb.

5. The apparatus recited in claim 3, wherein the body lumen comprises a urethra and a bladder and the connection comprises a surgical connection between the urethra and bladder, the first and third tubular members comprising a Foley catheter, the second tubular member comprising a concentric sleeve surrounding the Foley catheter.

6. The apparatus recited in claim 3, wherein the second tubular member comprises internal ribs that help maintain the second tubular member positioned concentric with the third tubular member, the ribs defining a plurality of axially extending grooves that help define the second conduit.

7. The apparatus recited in claim 1, wherein the second conduit is adapted to control the pressure in the space to create forces that act on the body lumen in directions that urge the surgical connection closed.

8. The apparatus recited in claim 1, wherein the second conduit is adapted to control the pressure to help prevent fluid leakage from the lumen through the surgical connection.

9. The apparatus recited in claim 1, wherein the second conduit is adapted to help maintain the pressure in the space below atmospheric pressure.

10. The apparatus recited in claim 1, wherein the second conduit is adapted to maintain the pressure in the space below the pressure in an abdominal cavity of the body.

11. The apparatus recited in claim 1, wherein the second conduit is adapted to maintain the pressure in the space below the pressure in a bladder of the body.

12. The apparatus recited in claim 1, wherein the surgical connection comprises a sutured connection.

13. The apparatus recited in claim 1, wherein the second conduit is adapted to apply a vacuum to the space.

14. The apparatus recited in claim 1, further comprising means for applying a vacuum to the second conduit.

15. A catheter system comprising:
a Foley catheter insertable through a urethra into a bladder, the Foley catheter having a proximal end portion for being positioned outside the urethra and an opposite distal end portion for being disposed in the bladder, the Foley catheter comprising a first conduit for emptying the bladder, an elastic bulb disposed on the distal end portion in the bladder, and a second conduit for delivering inflation fluid to the elastic bulb, the elastic bulb being inflatable to help retain the Foley catheter inserted in the bladder; and
a concentric tubular member encircling the Foley catheter, the tubular member having a proximal end portion positioned outside urethra and an opposite distal end portion that terminates before a proximal end of the elastic bulb, the tubular member defining a third conduit for controlling the pressure in a space defined between the tubular member and the bladder and urethra, a surgical connection between the bladder and urethra being exposed in the annular space.

16. The apparatus recited in claim 15, wherein the tubular member is adapted to control the pressure in the annular space to create forces that act on the body lumen in directions that urge the surgical connection closed.

17. The apparatus recited in claim 15, wherein the tubular member is adapted to control the pressure in the annular space to help prevent fluid leakage from the bladder and urethra through the surgical connection into the abdominal cavity.

18. The apparatus recited in claim 15, wherein the tubular member is adapted to help maintain the pressure in the annular space below atmospheric pressure.

19. The apparatus recited in claim 15, wherein the tubular member is adapted to maintain the pressure in the annular space below the pressure in an abdominal cavity of the body.

20. The apparatus recited in claim 15, wherein the tubular member is adapted to maintain the pressure in the space below the pressure in the bladder.

21. A method for facilitating the healing of a surgical connection in a body lumen, comprising the steps of:
providing a first conduit in the lumen;
positioning a distal end portion of the first conduit distally beyond the surgical connection, the first conduit directing fluid through the lumen and isolating the fluid from the surgical connection;
providing a second conduit in the lumen;
positioning a distal end portion of the second conduit adjacent the surgical connection; and
regulating the pressure in a space in which the surgical connection is positioned via the second conduit.

22. The method recited in claim 21, wherein:
the step of providing a first conduit comprises providing a first tubular member in the lumen, the first tubular member defining the first conduit;
the step of providing a second conduit comprises providing a second tubular member disposed in the first tubular member, the method further comprising the steps of:
providing a third tubular member disposed between the first and second tubular members, the second conduit being defined between the second and third tubular members, a third conduit being defined between the first and third tubular members, the third tubular member comprising an inflatable retention device disposed at a distal end of the third tubular member; and
delivering inflation fluid to the retention device via the third tubular member to inflate the retention device.

23. The method recited in claim 21, wherein the step of regulating the pressure comprises the step of maintaining the pressure in the space below atmospheric pressure.

24. The method recited in claim 21, wherein the step of regulating the pressure further comprises the step of maintaining the pressure in the space below the pressure in an abdominal cavity of the body.

25. The method recited in claim 21, wherein the step of regulating the pressure further comprises the step of maintaining the pressure in the space below the pressure in a bladder of the body.

26. The method recited in claim 21, wherein the step of regulating the pressure in the space comprises the step of regulating the pressure in the space to create forces that act on the body lumen in directions that urge the surgical connection closed.

27. The method recited in claim 21, wherein the step of regulating the pressure in the space comprises the step of regulating the pressure in the space to help prevent fluid leakage from the lumen through the surgical connection.

28. The method recited in claim 21, wherein the first and second conduits comprise portions of a catheter, the step of regulating the pressure in the space comprising the step of establishing and maintaining a vacuum in the space substantially throughout the duration of use of the catheter.
